(19) United States
(12) Patent Application Publication
(10) Pub. No.: US 2015/0007819 A1
(43) Pub. Date: Jan. 8, 2015

(54) APPARATUS, METHOD AND SYSTEM FOR A MOBILE MEDICAL DEVICE

(71) Applicants: Matthew MACONAUGHEY, Alexandria, VA (US); Marquis Dunton, Alexandria, VA (US)

(72) Inventors: Matthew MACONAUGHEY, Alexandria, VA (US); Marquis Dunton, Alexandria, VA (US)

(21) Appl. No.: 13/935,670

(22) Filed: Jul. 5, 2013

(51) Int. Cl. A61M 16/12; A61M 16/08; A61M 16/06; A61M 16/10; A61M 16/00; A61M 16/20; A61M 16/18

(52) U.S. Cl. CPC A61M 16/125; A61M 16/201; A61M 16/0833; A61M 16/18; A61M 16/1005; A61M 16/208; A61M 16/0003; A61M 16/06; A61M 16/106; A61M 2016/0027
USPC 128/203.25; 128/205.24; 128/205.12

(57) ABSTRACT

A portable medical device for sedating a patient includes a first vessel with a first substance contained under pressure and a second vessel with a second substance contained under pressure. A Y-valve may be provided with a first inlet, a second inlet, an outlet, a first pressure control valve between the first inlet and the outlet, and a second pressure control valve between the second inlet and the outlet. Tubes may connect the first and second vessels and a face mask to the Y-valve. The first pressure control valve may be opened to permit a fluid flow of the first substance from the first vessel to the face mask, and the second pressure control valve may be opened to permit a fluid flow of the second substance from the second vessel to the face mask.

### BACKGROUND

Surgical of other invasive medical procedures are well known in the medical arts to provide necessary and often vital care to an ailing person or patient. For the most part, surgical procedures are performed by highly trained professionals in a generally stable environment such as a hospital or other medical clinic or facility. However, in certain situations a surgical procedure may need to be performed in an atypical environment.

For example, in military zones, rural areas, storm shelters, survival shelters, or other remote locations, immediate access to medical care may be limited or unavailable. In emergency situations, time may be of the essence, and transporting the patient to a typical care facility may not be an option. Further, in many procedures it may be preferred or required that the patient be sedated. Sedation may provide temporary pain relief to the patient, as well as prevent or reduce sudden movements during the procedure. Therefore, having the ability to safely and effectively sedate a patient in a remote environment prior to a surgical procedure may be desirable.

### BRIEF DESCRIPTION OF THE INVENTION

In one exemplary embodiment, a portable medical device for sedating a patient includes a first vessel with a first substance contained under pressure and a second vessel with a second substance contained under pressure. A Y-valve may be provided with a first inlet, a second inlet, an outlet, a first pressure control valve between the first inlet and the outlet, and a second pressure control valve between the second inlet and the outlet. Tubes may connect the first and second vessels and a face mask to the Y-valve. The first pressure control valve may be opened to permit a fluid flow of the first substance from the first vessel to the face mask, and the second pressure control valve may be opened to permit a fluid flow of the second substance from the second vessel to the face mask.

In another exemplary embodiment, a portable medical device for sedating a patient includes a first vessel with a first substance contained under pressure, and at least one oxygen source. A Y-valve may be provided with a first inlet, a second inlet, an outlet, a first pressure control valve between the first inlet and the outlet, and a second pressure control valve between the second inlet and the outlet. Tubes may connect the first vessel, the oxygen source and a face mask to the Y-valve. The first pressure control valve may be opened to permit a fluid flow of the first substance from the first vessel to the face mask, and the second pressure control valve may be opened to permit a fluid flow of the oxygen source to the face mask.

In another exemplary embodiment, a method for sedating a patient for a procedure in a remote location may be provided. A first vessel containing a first sedative substance under pressure may be connected to a first inlet of a Y-valve. An oxygen source may be connected to a second inlet of a Y-valve. A first end of a surgical tube may be connected to an outlet of the Y-valve, and a second end of the surgical tube may be connected to a face mask. The face mask may be fitted over a nose and mouth of a patient. Pressure control valves may be opened to release a mixture of the first sedative substance and the oxygen through the outlet of the Y-valve. The mixture may be further provided through the surgical tube and into the face mask for inhalation by a patient.

### BRIEF DESCRIPTION OF THE DRAWINGS

Advantages of embodiments of the present invention will be apparent from the following detailed description of the exemplary embodiments. The following detailed description should be considered in conjunction with the accompanying figures in which:

FIG. 1 may show an exemplary embodiment of a portable medical apparatus.

FIG. 2 may show an exemplary embodiment of a portable medical apparatus.

### DETAILED DESCRIPTION OF THE INVENTION

Aspects of the present invention are disclosed in the following description and related figures directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention.

As used herein, the word “exemplary” means “serving as an example, instance or illustration.” The embodiments described herein are not limiting, but rather are exemplary only. It should be understood that the described embodiment are not necessarily to be construed as preferred or advantageous over other embodiments. Moreover, the terms “embodiments of the invention”, “embodiments” or “invention” do not require that all embodiments of the invention include the discussed feature, advantage or mode of operation.

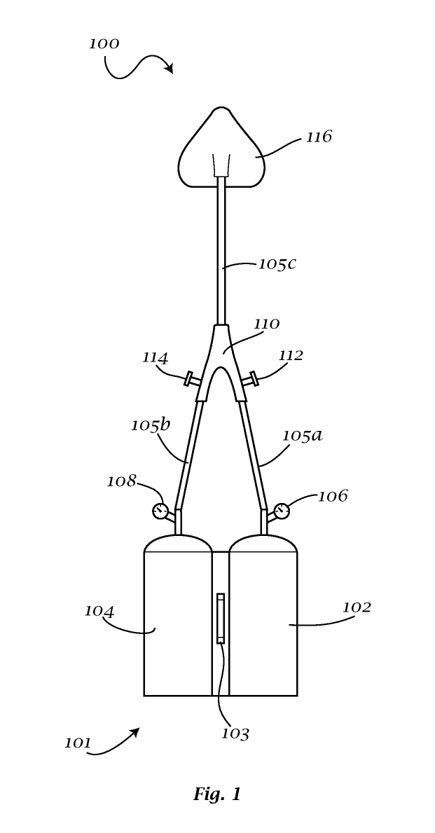

Turning now to FIG. 1, an exemplary embodiment of a portable medical apparatus 100 may be shown. Portable medical apparatus 100 may have a gas tank 101. Gas tank 101 may include an oxygen tank 102 and a nitrous tank 104, which may be separate storage vessels for containing a fluid such as a liquid or gas. In an exemplary embodiment, gas tank 101 may be formed as a single unit, but oxygen tank 102 and nitrous tank 104 may be separated which may prevent fluid communication or contamination between oxygen tank 102 and nitrous tank 104. Gas tank 101 may have at least one handle 103. At least one handle 103 may be, for example, a two sided handle that may allow a person to carry or otherwise transport portable medical apparatus 100.

Oxygen tank 102 may contain, for example, medical grade oxygen (O₂) held under pressure. Nitrous tank 104 may contain, for example, medical grade nitrous oxide (N₂O) held under pressure. In one exemplary embodiment, oxygen tank 102 may contain approximately one pound (1 lb.) of medical grade oxygen (O₂), and/or nitrous tank 104 may contain approximately two pounds (2 lbs.) of medical grade nitrous oxide (N₂O).

In still other embodiments, oxygen tank 102 and nitrous tank 104 may contain alternative substances and/or alternative weights of substances without departing from the scope of the present invention. For example, the portable medical apparatus 100 may be approximately 30-40 lbs. and arranged within a backpack for ease of portability. In another embodiment, the oxygen tank 102 and nitrous tank 104 may be, for example, 60 lb. liquid tanks and arranged within a medic's pack or a full sized field backpack as would be understood by a person of ordinary skill in the art. In still other exemplary embodiments a fixed medical apparatus similar to portable medical apparatus 100 may be provided that may be, for example, 100 lbs or more.

Oxygen tank 102 may have an outlet to connect to a first portion of a surgical tubing 105a. Nitrous tank 104 may have an outlet to connect to a second portion of surgical tubing 105b. Surgical tubing 105a-c may be, for example, surgical grade plastic or latex tubing or similar material as would be understood by a person of ordinary skill in the art. Surgical tubing 105a-c may be rigid or flexible. Surgical tubing 105a-c may have a length, for example, approximately 3-6 feet. In alternative embodiments, any portion of surgical tubing 105a-c may be customized to any length as would be understood by a person of ordinary skill in the art.

A first pressure gauge 106 may provide an instrument for measuring the pressure of the contents within oxygen tank 102. A second pressure gauge 108 may provide an instrument for measuring the pressure of contents within nitrous tank 104. First pressure gauge 106 and second pressure gauge 108 may provide, for example, a measurement in pounds per square inch (psi) and may provide a visual indication of the measurement to a user.

Portable medical apparatus 100 may have a Y-valve 110. Y-valve 110 may be, for example, a one way valve. A first end of Y-valve 110 may be in fluid communication with oxygen tank 102 by way of the first portion of surgical tubing 105a. A first pressure control valve 112 may be provided to selectively allow the passage of contents from oxygen tank 102 through Y-valve 110 to a third portion of surgical tubing 105c. A second end of Y-valve 110 may be in fluid communication with nitrous tank 104 by way of the second portion of surgical tubing 105b. A second pressure control valve 114 may be provided to selectively allow the passage of contents from nitrous tank 104 through Y-valve 110 to third portion of surgical tubing 105c. Thus, by opening both first pressure control valve 112 and second pressure control valve 114, a mixture of the contents from oxygen tank 102 and nitrous tank 104 may be provided to third portion of surgical tubing 105c.

Third portion of surgical tubing 105c may be in fluid communication with face mask 116. Face mask 116 may be, for example, a surgical mask that may be placed over the nose and mouth of a patient and secured to the patient as would be understood by a person of ordinary skill in the art. When the face mask 116 is secured to the patient, a supply of gas from at least one of oxygen tank 102 and nitrous tank 104 may be provided to the patient through Y-valve 110 and third portion of surgical 105c for inhalation by the patient. The content and supply of gas provided to the patient may be such that the patient may be temporarily sedated.

Still referring to FIG. 1, to use the portable medical apparatus 100, the oxygen tank 102 may be connected to the first pressure gauge 106, and the nitrous tank 104 may be connected to the second pressure gauge 108. First portion of surgical tubing 105a may be used to connect oxygen tank 102 and Y-valve 110. Second portion of surgical tubing 105b may be used to connect nitrous tank 104 and Y-valve 110. Third portion of surgical tubing 105c may be used to connect Y-valve 110 and face mask 116. The face mask 116 may be fitted over a patient's face.

The first pressure control valve 112 may be manipulated to allow the passage of contents from oxygen tank 102 through Y-valve 110 to the third portion of surgical tubing 105c and into face mask 116. The second pressure control valve 114 may also be manipulated to allow the passage of the contents from nitrous tank 104 through Y-valve 110 to third portion of surgical tubing 105c and into face mask 116. The patient may be instructed to breathe normally to inhale the gas mixture. Once the gas has produced the desired sedative effect, a procedure may be performed on the patient. The pressure and connection of medical apparatus 100 may be periodically monitored throughout the procedure.

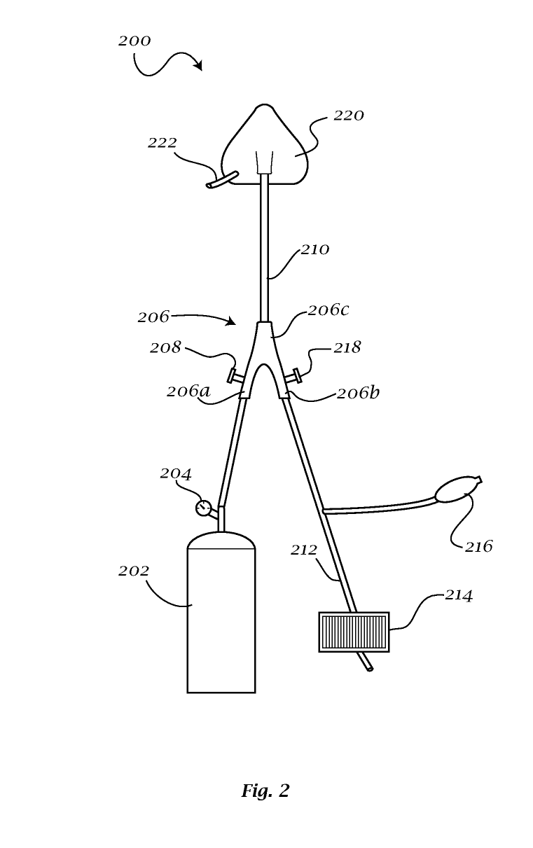

Turning now to FIG. 2, an exemplary embodiment of a portable medical apparatus 200 may be shown. Portable medical apparatus 200 may have a gas tank 202. Gas tank 202 may be a storage vessel for containing a fluid such as a liquid or gas under pressure. In an exemplary embodiment gas tank 202 may contain medical grade nitrous oxide (N₂O). Gas tank 202 may be connected to a first pressure gauge 204, which may provide an instrument for measuring the pressure of the contents within gas tank 202.

Gas tank 202 may be in fluid communication with Y-valve 206. Y-valve 206 may be, for example, a one way valve with a first inlet 206a, a second inlet 206b, and an outlet 206c. For example, gas tank 202 may be connected by a pipe, conduit, tube or the like to first inlet of valve 206a. A first valve 208 may be proximate the first inlet 206a. First valve 208 may be selectively opened or closed, for example, to allow the passage of contents from gas tank 202 into first inlet 206a, and through outlet 206c to tubing 210.

Portable medical apparatus 200 may have an air intake tube 212. Air intake tube 212 may be have a first end that is in fluid communication with atmospheric air and a second end in fluid communication with second inlet 206b. An air filter 214 may be proximate the first end of air intake tube 212 to filter air entering air intake tube 212. A pump 216 may be provided on air intake tube 212. Pump 216 may be, for example, a hand pump to control the intake of air from the atmosphere into air intake tube 212. In an exemplary embodiment, pump 216 may control the passage of air from the atmosphere into the first end of air intake tube 212, through air filter 214 and into second inlet 206b. In another exemplary embodiment, an air filtration canister may be attached to air intake tube 212.

A second valve 218 may be provided proximate the second inlet 206b. Second valve 218 may be selectively opened or closed, for example, to allow the passage of atmospheric air from the air intake tube 212 into second inlet 206b, through outlet 206c to tubing 210. Thus, by opening both first valve 208 and second valve 218, a mixture of the contents from gas tank 202 and filtered atmospheric air from air intake tube 212 may be provided to tubing 210.

Tubing 210 may be, for example, surgical grade tubing. Tubing 210 may have a first end coupled to and in fluid communication with outlet 206c, and a second end coupled to and in fluid communication with face mask 220. Tubing 210 may be rigid or flexible. Tubing 210 may have a length, for example approximately 3-6 feet. In alternative embodiments, the length of tubing 210 may be customized to any length as would be understood by a person of ordinary skill in the art.

Face mask 220 may be, for example, a surgical mask that may be placed over the nose and mouth of a patient and secured to the patient as would be understood by a person of ordinary skill in the art. When face mask 220 is secured to the patient, a mixture of gas from gas tank 202 and filtered atmospheric air from air intake tube 212 may be provided to the patient from outlet 206c and tubing 210 for inhalation by the patient. The content and supply of gas provided to the patient may be such that the patient may be temporarily sedated. Face mask 220 may have an exhalation exit 222. Exhalation exit 222 may be, for example, a one way valve that may permit exhaled air to exit face mask 220 into the atmosphere, while preventing additional intake of atmospheric air into face mask 220.

Still referring to FIG. 2, to use portable medical apparatus 200, the gas tank 202 may be connected to the first pressure gauge 204. Gas tank 202 may be configured in fluid communication with first inlet 206a. Air intake tube 212 may be configured in fluid communication with second inlet 206b. Tubing 210 may be configured in fluid communication with outlet 206c and face mask 220. Face mask 220 may be secured over the mouth and nose of a patient.

Proximate the first inlet 206a, the first valve may be opened to allow the passage of pressurized contents from gas tank 202 into first inlet 206a, and through outlet 206c to tubing 210. Proximate the second inlet 206b, the second valve may be opened. Pump 216 may be used to draw atmospheric air into air intake tube 212 and through air filter 214. Filtered air may then pass from air intake tube into second inlet 206b, and through outlet 206c to tubing 210.

The gas mixture may travel through tubing 210 to face mask 220. The patient may be instructed to breathe normally to inhale the gas mixture. Exhaled air may pass through exhalation exit 222 to the atmosphere. Once the gas has produced the desired sedative effect, a procedure may be performed on the patient. The pressure and connection of medical apparatus 200 may be periodically monitored throughout the procedure.

The medical apparatus of the present invention may be scaled for any emergency or non-emergency situation. For example a larger version may be used by first responders in temporary emergency medical stations. For example, the medical apparatus may be used during or after a natural or man-made disaster such as a hurricane, tornado, earthquake, terrorist attack, nuclear event or the like. In other exemplary embodiments, the medical apparatus of the present invention may be suitable for mounting in, for example, a helicopter or ambulance.

In one exemplary embodiment, the medical apparatus of the present invention may be provided with a bullet resistant hard case. In still other exemplary embodiments, the tanks of the medical apparatus may be provided with individual tank sleeves that may be, for example, bullet resistant, temperature resistant or the like. Accordingly, the present invention may be protected from damage during any potential situation.

The medical apparatus of the present invention may be very simple to operate and require minimal training. For example, an anesthesiology or other medical license may not be required to operate the medical apparatus of the present invention.

In some exemplary embodiments, a plurality of additional tanks may be utilized with the present invention as would be understood by a person of ordinary skill in the art. For example, a third tank, fourth tank etc., with different substances may also be included. In these exemplary embodiments, a variety of gas mixtures in various concentrations may be provided by controlling the fluid flow from each of the plurality of tanks. In still other exemplary embodiments, the contents from only a single tank from the plurality of tanks may be provided. For example, in some medical procedures such as a treatment a collapsed lung or a tracheotomy, a patient may only require pure oxygen. In these situations, the fluid flow from all other tanks may be restricted such that only oxygen is provided to the patient.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A portable medical apparatus, comprising:
a first vessel with a first substance contained under pressure;
a second vessel with a second substance contained under pressure, the second vessel coupled to the first vessel;
a Y-valve with a first inlet, a second inlet, an outlet, a first pressure control valve between the first inlet and the outlet, and a second pressure control valve between the second inlet and the outlet;
a first tube coupled between the first vessel and the first inlet;
a second tube coupled between the second vessel and the second inlet;
a third tube coupled between the outlet and a face mask configured to seal over at least one of a nose and mouth of a user,
wherein the first pressure control valve is opened to permit a fluid flow of the first substance from the first vessel to the face mask, and the second pressure control valve is opened to permit a fluid flow of the second substance from the second vessel to the face mask.

2. The portable medical apparatus of claim 1, wherein the first substance is medical grade nitrous oxide, and the second substance is medical grade oxygen.

3. The portable medical apparatus of claim 1, further comprising:
at least one pressure gauge that provides a pressure reading for the first substance contained in the first vessel.

4. The portable medical apparatus of claim 1, further comprising:
at least one pressure gauge that provides a pressure reading for the second substance contained in the second vessel.

5. The portable medical apparatus of claim 1, wherein the first vessel and the second vessel are formed as a single unit.

6. The portable medical apparatus of claim 1, further comprising:
at least one handle attached to at least one of the first vessel and the second vessel.

7. The portable medical apparatus of claim 1, wherein the Y-valve is a one way valve between the first inlet and the outlet and between the second inlet and the outlet.

8. The portable medical apparatus of claim 1, wherein the first substance and the second substances are at least one of a gas and a liquid.

9. The portable medical apparatus of claim 1, wherein the first tube, the second tube, and the third tube are surgical tubes.

10. The portable medical apparatus of claim 1, wherein the first pressure control valve is closed to stop a fluid flow of the first substance from the first vessel to the face mask, and the second pressure control valve is closed to stop a fluid flow of the second substance from the second vessel to the face mask.

11. A portable medical apparatus, comprising:
a first vessel containing a first substance under pressure;
a Y-valve with a first inlet, a second inlet, an outlet, a first pressure control valve between the first inlet and the outlet, and a second pressure control valve between the second inlet and the outlet;
a first tube coupled between the first vessel and the first inlet;
a second tube with a first end in fluid communication with atmospheric air, and a second end coupled to the second inlet;
a third tube coupled between the outlet and a face mask configured to seal over at least one of a nose and mouth of a user;
wherein the first pressure control valve is opened to permit a fluid flow of the first substance from the first vessel to the face mask, and the second pressure control valve is opened to permit a fluid flow of atmospheric air to the face mask.

12. The portable medical apparatus of claim 11, wherein the first substance is medical grade nitrous oxide.

13. The portable medical apparatus of claim 11, further comprising:
an air filter within the second tube and configured to filter atmospheric air between the first end and the second end.

14. The portable medical apparatus of claim 11, further comprising:
a pump operable to draw atmospheric air into the second tube.

15. The portable medical apparatus of claim 11, further comprising:
a one way exhalation tube coupled to the face mask to allow the release of exhaled air to the atmosphere.

16. A method of sedating a patient for a procedure in a remote location, comprising the steps of:
connecting a first vessel containing a first sedative substance under pressure to a first inlet of a Y-valve, the first inlet having a first pressure control valve between the first inlet and an outlet of the Y-valve;
connecting an oxygen source to a second inlet of a Y-valve, the first inlet having a first pressure control valve between the second inlet and the outlet of the Y-valve;
connecting a first end of a surgical tube to the outlet, and a second end of the surgical tube to a face mask;
fitting the face mask over a nose and mouth of a patient;
opening the first and second pressure control valves to release a mixture of the first sedative substance and the oxygen through the outlet of the Y-valve and the surgical tube and into the face mask for inhalation by the patient.

17. The method of claim 16, further comprising the step of:
using atmospheric air as the oxygen source; and
filtering the atmospheric air prior to the second inlet.

18. The method of claim 16, further comprising the step of:
providing a one-way exhalation valve on the face mask to allow the release of exhaled gas to the atmosphere.

19. The method of claim 16, further comprising the step of:
checking a pressure reading of the sedative substance in the first vessel, and further checking the connections at the Y-valve and the face mask during a procedure.

20. The method of claim 16, further comprising the step of:
providing at least one handle on the first vessel; and
carrying the first vessel from a first location to a second location.
